(19) United States
(12) Patent Application Publication
(10) Pub. No.: US 2023/0122168 A1
(43) Pub. Date: Apr. 20, 2023

(54) Conformal Inference for Optimization

(71) Applicant: Flagship Pioneering Innovations VI, LLC, Cambridge, MA (US)

(72) Inventors: Molly Krisann Gibson, Medford, MA (US); Kevin Kaichuang Yang, Arlington, MA (US); Maxim Baranov, Medford, MA (US); Andrew Lane Beam, Jamaica Plain, MA (US)

(21) Appl. No.: 17/759,838

(22) PCT Filed: Jan. 29, 2021
(86) PCT No.: PCT/US2021/015848

§ 371 Date: Jul. 29, 2022

Related U.S. Application Data
(60) Provisional application No. 62/967,941, filed on Jan. 30, 2020.

(51) Int. Cl. G16B 30/00; G16B 40/00

(52) U.S. Cl. CPC G16B 30/00; G16B 40/00

(57) ABSTRACT

Accurate function estimations and well-calibrated uncertainties are important for Bayesian optimization (BO). Most theoretical guarantees for BO are established for methods that model the objective function with a surrogate drawn from a Gaussian process (GP) prior. GP priors are poorly-suited for discrete, high-dimensional, combinatorial spaces, such as biopolymer sequences. Using a neural network (NN) as the surrogate function can obtain more accurate function estimates. Using a NN can allow arbitrarily complex models, removing the GP prior assumption, and enable easy pretraining, which is beneficial in the low-data BO regime. However, a fully-Bayesian treatment of uncertainty in NNs remains intractable, and existing approximate methods, like Monte Carlo dropout and variational inference, can highly miscalibrate uncertainty estimates. Conformal Inference Optimization (CI-OPT) uses confidence intervals calculated using conformal inference as a replacement for posterior uncertainties in certain BO acquisition functions. A conformal scoring function with properties amenable for optimization is effective on standard BO datasets and real-world protein datasets.

### RELATED APPLICATION

This application claims the benefit of U.S. Provisional Application No. 62/967,941, filed on Jan. 30, 2020. The entire teachings of the above application(s) are incorporated herein by reference.

### BACKGROUND

Machine learning commonly employs statistical models that computer-implemented methods can leverage to perform given tasks. Often, the statistical models employed by machine learning methods detect patterns, and use said patterns to predict future behavior. The statistical models and neural networks employed by machine learning methods are typically trained with real-world data, and the machine learning methods leverage said real-world data to predict the future behavior.

### SUMMARY

Accordingly, there is a need for improved machine learning models that provide better predictions of data using less training data. Accurate function estimations and well-calibrated uncertainties are important for Bayesian optimization (BO). Most theoretical guarantees for BO are established for methods that model the objective function with a surrogate drawn from a Gaussian process (GP) prior. GP priors are poorly-suited for discrete, high-dimensional, combinatorial spaces, such as biopolymer sequences. Using a neural network (NN) as the surrogate function can obtain more accurate function estimates. Using a NN can allow arbitrarily complex models, removing the GP prior assumption, and enable easy pretraining, which is beneficial in the low-data BO regime. However, a fully-Bayesian treatment of uncertainty in NNs remains intractable, and recent results have shown that approximate inference can result in estimates that poorly reflect the true posterior. Conformal Inference Optimization (CI-OPT) uses confidence intervals calculated using conformal inference as a replacement for posterior uncertainties in certain BO acquisition functions. While current methods do not combine conformal inference with BO due to their intractability, Applicant discloses a conformal scoring function with properties amenable for optimization that is effective on synthetic optimization tasks, standard BO datasets and real-world protein datasets.

In an embodiment, a computer-implemented method for optimizing design of biopolymer sequences can include training a machine learning model using an observed biopolymer sequences and labeled biopolymer sequences corresponding to each observed biopolymer sequence. A labeled sequence is a sequence associated with a real number measuring some property of interest. The method can further include determining a candidate biopolymer sequences to observe having a highest predicted value of the labeled biopolymer sequences based on the machine learning model. Candidate biopolymer sequences can include either known sequences (e.g., previously encountered, previously observed, or natural sequences) or newly designed sequences. The method can further include, for each candidate biopolymer sequence, determining a conformal inference interval representing a likelihood that the candidate biopolymer sequence has the predicted value of the labeled biopolymer sequences. The method can further include selecting at least one candidate biopolymer sequence having an optimized linear combination of the conformal inference interval and the predicted value of the labeled biopolymer sequences.

In an embodiment, the value of a labeled sequence is number being used as its label as described above. Therefore, a predicted value of a sequence is the predicted label of the sequence. A person having ordinary skill in the machine learning art can appreciate such a definition of label. The sequence or data points are the machine learning input (x), and the prediction/measurement/optimization is the label (y).

In embodiments, the conformal inference interval includes a center value and an interval range. The center value can be a mean value.

In embodiments, the machine learning model is a neural network fine-tuned using the observed biopolymer sequences and their labels. A fine-tuned neural network is a neural network that is pretrained on a large dataset that uses those weights as initial weights for a smaller dataset. Fine tuning can speed up training and overcome a small data set size. In an embodiment, determining the conformal inference interval is based on a second set of observed biopolymer sequences. The second set of sequences are the set of sequences used to tune the conformal scores.

In an embodiment, determining the conformal inference interval can further include calculating a residual interval based on each output of the machine learning model for the second set of observed biopolymer sequences and corresponding labeled biopolymer sequences corresponding to each of the second set of biopolymer sequences. Determining the conformal inference interval can further include for each output of the machine learning model, calculating an average distance to nearest neighbors of the observed biopolymer sequences within the metric space. Determining the conformal inference interval can further include calculating a conformal score based on a ratio of the residual to a sum of the average distance and a constant. As described below, a metric space is a set of possible sequences. An example of a metric can be Levenshstein distance. In embodiments, the constant can change in each iteration.

In an embodiment, selecting the at least one candidate biopolymer sequence includes calculating an average distance in a metric space to a nearest neighbors in the metric space, generating a confidence interval based on the at least one candidate biopolymer sequence and the average distance, and selecting a candidate biopolymer sequence based on the confidence interval.

In embodiments, the conformal interval can be at least 50% and at most 99%. The biopolymer sequence can include at least one of an amino acid sequence, a nucleic acid sequence, and a carbohydrate sequence. The nucleic acid sequence can be a deoxyribonucleic acid (DNA) sequence or ribonucleic acid (RNA) sequence. The amino acid sequence can be any sequence, encompassing all proteins such as, for example, enzymes, growth factors, cytokines, hormones, signaling proteins, structural proteins, kinetic proteins, antibodies (including both immunoglobulin-based molecules and alternative molecular scaffolds), and combinations of the foregoing, including fusion proteins and conjugates.

In an embodiment, a computer computer-implemented method for optimizing design of biopolymer sequences and corresponding system can include training a model to approximate labeled biopolymer sequences of initial samples from a plurality of observed sequences. The method can further include, for a particular batch of the plurality of observed sequences, having labeled biopolymer sequences generated by a trained model and conformal interval for each observed sequence, choosing at least one sequence from the plurality of observed sequences that optimizes a combination of the labeled biopolymer sequences generated by the trained model and the conformal interval. The method can further include recalculating the conformal interval for the remaining sequences.

In embodiments, the method can further include repeating choosing the at least one sequence and recalculating the conformal interval for each of a plurality of batches. In embodiments, the method can further include identifying an optimal number of batch experiments to run in parallel. In embodiments, identifying can be based on optimizing wet-lab resources.

In an embodiment, a computer-implemented method can include training a machine learning model using data points within a metric space and functional values corresponding to each observed data point. The functional value(s) are real number(s) measuring some property of interest of the data points. The method can further include determining a candidate data points to observe having a highest predicted functional value based on the machine learning model. Candidate data points can include either known data points (e.g., previously encountered, previously observed, or natural data points) or newly designed data points. The method can further include, for each candidate data point, determining a conformal inference interval representing a likelihood that the candidate data point has the predicted functional value of the data points. The method can further include selecting at least one candidate data points having an optimized linear combination of the conformal inference interval and the predicted functional value of the data points. A person having ordinary skill in the art can recognize that the data points can include images, video, audio, other media, and other data that can be interpreted by a machine learning model.

In an embodiment, a computer computer-implemented method and corresponding system can include training a model to approximate functional values data points of initial samples from a plurality of observed data points. The method can further include, for a particular batch of the plurality of observed data points, having functional values generated by a trained model and conformal interval for each observed data point, choosing at least one sequence from the plurality of the data points that optimizes a combination of the labeled data points generated by the trained model and the conformal interval. The method can further include recalculating the conformal interval for the remaining data points.

In embodiments, a computer-implemented method for optimizing design based on a distribution of data includes training a machine learning model using a plurality of observed data and labeled data corresponding to each observed data. The method further includes determining a plurality of candidate data to observe having a highest predicted value of the labeled data based on the machine learning model. The method further includes, for each candidate data, determining a conformal inference interval representing a likelihood that the candidate data has the predicted value of the labeled data. The method further includes selecting at least one candidate data having an optimized linear combination of the conformal inference interval and the predicted value of the labeled data.

In embodiments, the above methods further include providing the at least one selected biopolymer sequence to a means for synthesizing the selected biopolymer sequence, optionally wherein the at least one selected biopolymer sequence is synthesized.

In embodiments, the method further includes synthesizing the at least one selected biopolymer sequence.

In embodiments, the method further includes assaying the at least one selected biopolymer sequence (such as in a qualitative or quantitative chemical assay).

In embodiments, a non-transitory computer readable medium is configured to store instructions for optimizing design of biopolymer sequences thereon. The instructions, when executed by a processor, cause the processor to train a machine learning model using a plurality of observed biopolymer sequences and labeled biopolymer sequences corresponding to each observed biopolymer sequence, determine a plurality of candidate biopolymer sequences to observe having a highest predicted value of the labeled biopolymer sequences based on the machine learning model, determine, for each candidate biopolymer sequence, a conformal inference interval representing a likelihood that the candidate biopolymer sequence has the predicted value of the labeled biopolymer sequences, and select at least one candidate biopolymer sequence having an optimized linear combination of the conformal inference interval and the predicted value of the labeled biopolymer sequences.

In embodiments, a system for optimizing design of biopolymer sequences includes a processor and a memory with computer code instructions stored thereon. The processor and the memory, with the computer code instructions, are configured to cause the system to train a machine learning model using a plurality of observed biopolymer sequences and labeled biopolymer sequences corresponding to each observed biopolymer sequence, determine a plurality of candidate biopolymer sequences to observe having a highest predicted value of the labeled biopolymer sequences based on the machine learning model, determine, for each candidate biopolymer sequence, a conformal inference interval representing a likelihood that the candidate biopolymer sequence has the predicted value of the labeled biopolymer sequences, and select at least one candidate biopolymer sequence having an optimized linear combination of the conformal inference interval and the predicted value of the labeled biopolymer sequences.

In embodiments, disclosed herein are one or more selected biopolymer sequences that are obtainable by the method of any one of the preceding claims.

In embodiments, the one or more selected biopolymer sequences are manufactured by an in vitro method of chemical synthesis. In other embodiments, the one or more selected biopolymer sequences are manufactured by biosynthesis, e.g., using a cell-based system, such as a bacterial, fungal, or animal (e.g., insect or mammalian) system. For example, in some embodiments, the one or more selected biopolymer sequences are one or more selected polypeptide sequences. In certain more particular embodiments, the one or more selected polypeptide sequences are manufactured by chemical synthesis, e.g., on a peptide synthesizer. In other more particular embodiments, the one or more selected biopolymer sequences are synthesized by a biological system, e.g., comprising steps of providing one or more nucleic acid sequences (e.g., in an expression vector) to a biological system (e.g., a host cell or in vitro translation system, such as a transcription and translation system), culturing the biological system under conditions to promote synthesis of the one or more selected polypeptide sequences, and isolating the synthesized one or more selected polypeptide sequences from the system.

In embodiments, a composition includes the one or more selected biopolymer sequences optionally containing a pharmaceutically acceptable excipient.

In embodiments, a method includes contacting the composition or selected biopolymer sequences of any one of the preceding claims with one or more of: a test compound, a biological fluid, a cell, a tissue, an organ, or an organism.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A and 1B are graphs illustrating results for sequential optimization on two synthetic tasks.

FIGS. 1C and 1E are graphs illustrating results for sequential optimization on protein datasets.

FIGS. 1D and 1F are graphs illustrating similar results for batch optimization on the protein datasets.

FIGS. 1G-1I compare uncertainties calculated from a GP posterior, conformal inference with a neural network residual estimator, and conformal inference with scaled k-nearest neighbors.

FIG. 2 is a flow diagram illustrating an example embodiment calculating conformal intervals for predictions from a fine-tuned neural network using nearest-neighbors in sequence space as in the present disclosure.

FIG. 3 is a flow diagram illustrating an example embodiment of a method of batch optimization using the above conformal intervals.

FIG. 4 is a flow diagram illustrating an example embodiment of the present disclosure.

FIG. 5 is a flow diagram illustrating an example embodiment of the present disclosure.

FIG. 6 illustrates a computer network or similar digital processing environment in which embodiments of the present invention may be implemented.

FIG. 7 is a diagram of an example internal structure of a computer (e.g., client processor/device or server computers) in the computer system of FIG. 6.

### DETAILED DESCRIPTION

A description of example embodiments follows.

Bayesian optimization (BO) is a popular technique for optimizing black-box functions. BO's applications include, among others, experimental design, hyperparameter tuning, and control systems. Traditional BO methods rely on well-calibrated uncertainties from a posterior induced by observations of an objective function or true function. The objective function is a property to be optimized. For example, if the system is optimizing biopolymers, the objective function may optimize a property of the biopolymers. Using uncertainty to guide decisions makes BO especially powerful in the low-data situations. Current implementations, such as those shown in “Deep Bayesian Bandits Showdown: An Empirical Comparison of Bayesian Deep Networks for Thompson Sampling” by Riquelme et al. in arXiv preprint arXiv:1802.09127, (2018) (hereinafter “Riquelme”), show that both accurate function estimations and well-calibrated uncertainties are important for strong performance on real-world problems.

Most theoretical guarantees for BO are established for methods that model the objective function with a surrogate drawn from a Gaussian process (GP) prior. If the function strongly violates the GP prior, the resulting posterior probabilities can be poor estimates of the true function, have miscalibrated uncertainties, or both. This is especially important when the design space is discrete and combinatorial (e.g., a biopolymer sequence such as a protein sequence), as most GP priors are designed for low-dimensional continuous spaces and may not be good surrogates for these types of spaces.

One way to obtain more accurate function estimates is to use a neural network as the surrogate function. A surrogate function is a function that models the objective/true function. In addition to allowing arbitrarily complex models and removing the GP prior assumption, using a neural network enables pretraining, which can be especially beneficial in the low-data BO regime. However, a fully-Bayesian treatment of uncertainty in neural networks, such as using Hamiltonian Monte Carlo to estimate the posterior remains computationally intractable, and recent results have shown that approximate inference can result in estimates that poorly reflect the true posterior. One alternative is to use a Bayesian linear regression on top of a neural network as the surrogate function. The methods of Riquelme compare the performance of different approximate Bayesian uncertainty quantification methods on BO tasks.

Conformal inference can collectively refer to a family of uncertainty quantification methods. Conformal inference methods provide valid, calibrated prediction intervals under the assumption that the data are exchangeable. A person having ordinary skill in the art can recognize that exchangeable data can be consistent with the equation p(x₁, x₂, . . . xₙ)=p(xₛ₁, xₛ₂, . . . , xₛₙ) for any permutation of the respective indices. Unlike Bayesian methods such as GP models, conformal inference does not rely on strong underlying assumptions about the data or the target function. Conformal inference can also be applied on top of any machine-learning model that can allow valid prediction intervals to be built on top of modern deep-learning technology, such as large pre-trained models for which Bayesian inference would be intractable.

In an embodiment of the present disclosure, a method and corresponding system employs conformal confidence intervals with Bayesian optimization methods. The combination of conformal confidence intervals with Bayesian optimization methods are referred to below as Conformal Inference Optimization (CI-OPT). CI-OPT employs confidence intervals that are calculated using conformal inference as a drop-in replacement for posterior uncertainties in certain BO acquisition functions.

At a high level, the problem to be solved can be described as finding the maximum of some function ƒ(x) over some decision set [symbol] is a first goal. The true function ƒ(x) is unknown, however, function evaluations are known, but said functional evaluations are possibly noisy. The more valuable function evaluations are expensive to calculate, therefore maximizing ƒ with as few function evaluations as possible is desired. For instance, consider ƒ being a function representing fitness of a protein sequence. Additionally, evaluating a batch of query points in parallel may be far less expensive computationally than evaluating the same queries sequentially.

Current Bayesian optimization approaches and methods, as described further in “Taking the Human out of the Loop: A Review of Bayesian Optimization” by Shahriari et al. in Proceedings of the IEEE, 104(1): 148-175 (2015) (hereinafter “Shahriari”), begin by placing a prior on ƒ. At time step t+1, the possibly noisy previous observations Yₜ={y₁, . . . , yₜ} at locations Xₜ={x₁, . . . , xₜ} induce a posterior distribution for ƒ. An acquisition function a(x, t) determines what point in χ to query next via a proxy optimization xₜ₊₁=argmaxₓa(x|Dₜ, t), where Dₜ={Xₜ, yₜ}. The acquisition function uses the posterior over ƒ to balance exploiting information gained from previous queries and exploring regions with high uncertainty.

Gaussian processes are a common choice for the function prior (see, e.g., Williams et al., “Gaussian processes for machine learning,” Volume 2. MIT press Cambridge, Mass., 2006) (hereinafter “Williams”). GPs are infinite collections of random variables such that every finite subset of random variables has a multivariate Gaussian distribution. A GP model assumes that the unknown true function is drawn from a GP prior, and then the GP model uses the observations to calculate a posterior over functions. A key advantage of GP models is that there is a simple closed-form solution for the posterior, which makes them one of the most popular theoretical tools for Bayesian optimization. The GP posterior at each step can be marginalized in closed-form to arrive at a predictive mean μₜ(x) and standard deviation σₜ(x) for x∈ [symbol] .

Notable acquisition functions include:
a) expected improvement, as shown by “Efficient Global Optimization of Expensive Black-Box Functions” by Jones et al. in Journal of Global optimization, 13(4):455-492, 1998. (hereinafter “Jones”):

a_{EI}(x|Dₜ,t)= [symbol] _{ƒ(x|D)}[max(ƒ(x)−ƒ(x*),0)] (Eq. 1)

where ƒ(x*) is the best (e.g., maximum) evaluation observed in Dₜ;
a) Gaussian Process Upper Confidence Bound (GP-UCB) as shown in “Gaussian Process Optimization in the Bandit Setting: No Regret and Experimental Design.” by Srinivas et al. in arXiv preprint arXiv:0912.3995, 2009 (hereinafter “Srinivas”):

a_{UCB}(x|Dₜ,t)=μₜ(x)+βₜσₜ(x) (Eq. 2)

where βₜ is a tunable hyperparameter that controls the tradeoff between exploration and exploitation; and
a) Gaussian process mutual information (GP-MI) as shown in “Gaussian Process Optimization with Mutual Information” by Contal et al. in International Conference on Machine Learning, pp. 253-261, 2014. (hereinafter “Contal”), which is less prone to overexploration than GP-UCB:

a_{MI}(x|Dₜ,t)=μₜ(x)+α(\sqrt{σₜ²(x)+\hat{γ}ₜ}−\sqrt{\hat{γ}ₜ}) (Eq. 3)

where α is a tunable hyperparameter and \hat{γ}ₜ=Σᵢ₌₁^{t}σᵢ²(xᵢ).

More generally, observations may be queried in batches instead of strictly sequentially. In the batch setting, at time t+1, a set of B items xᵣ, . . . , x_{r+B-1} are selected to be queried based on (possibly noisy) previous observations yₜ={y₁, . . . , yₜ} a at locations Xₜ={x₁, . . . , xₜ}. In general, B can be chosen adaptively at every iteration (see, e.g., “Parallelizing exploration-exploitation tradeoffs in gaussian process bandit optimization” by Desautels et al. in Journal of Machine Learning Research, 15:3873-3923, 2014. (hereinafter “Desautels”), but in this example explores settings in which the batch size is fixed. Many batch Bayesian optimization methods use uncertainty on the acquisition function to generate appropriately diverse batches.

For example, the methods of Desautels generalize the GP-UCB acquisition function to batch queries by updating t after each selection within a batch as if that selection had been queried and observed to be its mean posterior value. Alternatively, acquisition functions can be sampled from the GP posterior to generate diverse batches, as shown in “Maximizing Acquisition Functions for Bayesian Optimization” by Wilson in Advances in Neural Information Processing Systems, pp. 9884-9895, 2018 (hereinafter “Wilson”) and “Sampling Acquisition Functions for Batch Bayesian Optimization” by De Palma et al. in arXiv preprint arXiv:1903.09434, 2019 (hereinafter “De Palma”).

Conformal inference is an auxiliary method that provides exact, finite sample 1−ϵ prediction intervals for any underlying machine-learning model, as shown in “Transduction with Confidence and Credibility” by Saunders et. al., 1999 (hereinafter “Saunders”). Given exchangeable samples z₁, . . . , zₙ∈ [symbol] × [symbol] , , a desired confidence level ε, and some conformal scoring function C:Z→ [symbol] conformal inference methods evaluate C(z₁), C(z₂) . . . , C(zₙ) and then set cₛ to be the (1−ε) percentile score. As such, it is a probability 1−ε that a test example z_{*} was drawn from [symbol] if C(z_{*})<cₛ. A series of random variables z₁, z₂, z₃ . . . is exchangeable if for any finite permutation σ of the indices P(z₁, z₂, z₃ . . . )=P(z_{σ(1)}, z_{σ(2)}, z_{σ(3)} . . . ).

Conformal regression, as shown in “Regression Conformal Prediction With Nearest Neighbours” by Papadopoulos et al. in Journal of Artificial Intelligence Research, 40:815-840, 2011 (hereinafter “Papadopoulos”), aims to find heteroskedastic confidence intervals. In an example, consider a regressor h: χ→ [symbol] trained on Zₜᵣ={Xₜᵣ, Yₜᵣ}, and a desired significance level ε∈(0,1). A conformal score can then be calculated for each element in a conformal training set Z_{c}={X_{c}, Y_{c}} (disjoint from Zₜᵣ), which ideally are disjoint from the data used to train h, using a conformal function C of the following form:

$$C ( x , y ) = \frac{\vert h ( x ) - y \vert}{ℊ ( x ) + β} \qquad ( Eq . 4 )$$

where g(x) is a function that measures the difficulty expected of the true function ƒ(x) to be able to predict, and where β is a hyperparameter that controls the sensitivity to g. From C, conformal inference methods can calculate calibration conformal scores c for items in Z_{c}, and let cₛ be the (1−ε)-percentile calibration score. The prediction region for a new sample x_{*} is then

Γ(x_{*})=h(x_{*})±cₛ[g(x_{*})+β] (Eq. 5)

and contains y_{*} with probability 1−ε. Notably, the intervals generated are valid for any g, although they may be too broad or too uniform to be useful.

An exemplary conformal scoring function C, having properties amenable for optimization, is described herein. Conformal prediction intervals (e.g., a 95% conformal interval) can be used as a drop-in replacement for t in a Bayesian optimization style procedure. However, a person having ordinary skill in the art can understand that other conformal intervals can be used. In an example, conformal intervals ranging inclusively from 50%-99% can be used.

At step t, a regressor h is trained on {Xₜ, yₜ}, a conformal scoring function g, sensitivity parameter β, and 95 percentile calibration score cₛ. Then, the equations

$${μ}_{t , CI} ( {x}_{*} ) = h ( {x}_{*} ) \qquad ( Eq . 6 )$$

$$and$$

$${σ}_{t , CI} ( {x}_{*} ) = \frac{1}{2} {c}_{s} [ ℊ ( {x}_{*} ) + β ] \qquad ( Eq . 7 )$$

replace μₜ and σₜ in the UCB (Equation 2) or MI (Equation 3) acquisition functions.

Choosing g is crucial for inducing intervals that balance exploration and exploitation. Ideally, intervals should be narrower in regions that have been densely sampled. For example, a common method is for g to be a model trained to predict the residuals h(x) y| for x, y∈Z_{c}. This g essentially uses Z_{c} to directly learn where the intervals should be narrower or wider but does not explicitly account for epistemic uncertainty caused by under-sampling certain regions of [symbol] . Therefore, g can be set as the average distance to the k nearest neighbors of x in Xₜᵣ:

$${ℊ}_{kNN} ( x ) = \frac{1}{k} {\sum }_{i = 1}^{k} d ( x , {x}_{tr , i} ) \qquad ( Eq . 8 )$$

where x_{tr,i} is the i^{th} nearest neighbor of x in the training set. In practice, scaling g during conformal training improves the stability of the intervals, and is a novel conformal scoring function.

$${\overline{ℊ}}_{kNN} ( x ) = {ℊ}_{kNN} ( x ) \frac{{max}_{{Z}_{c}} \vert h ( x ) - y \vert}{{max}_{{Z}_{c}} ℊ ( x )} \qquad ( Eq . 9 )$$

Intuitively, this can be related to two sources of uncertainty in a Guassian process posterior. The residual |h(x)−y| is analagous to homoscedastic noise variance, while g_{kNN} is analogous to a hard-thresholded stationary GP covariance function. In other words, Eq. 9 includes a conformal score that explicitly estimates epistemic uncertainty.

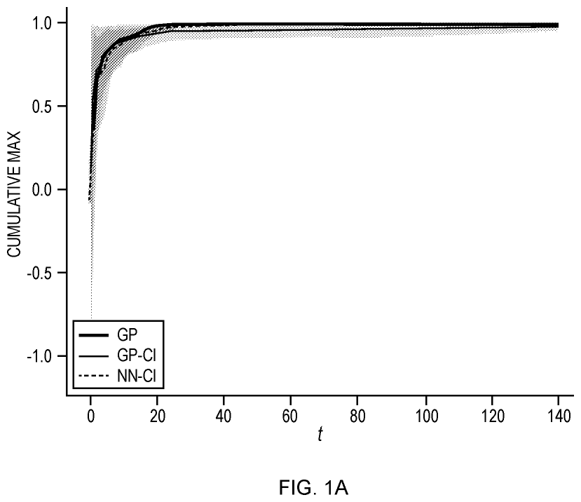

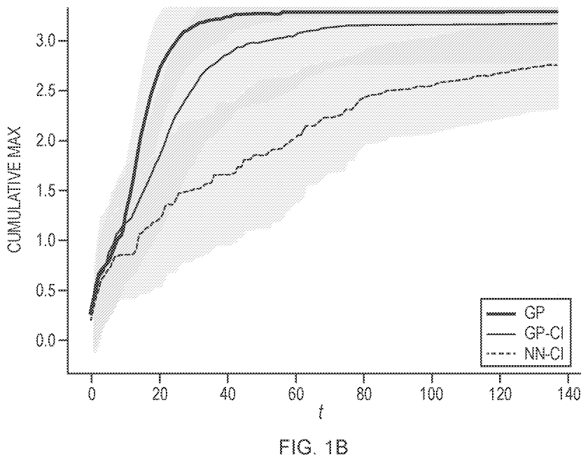

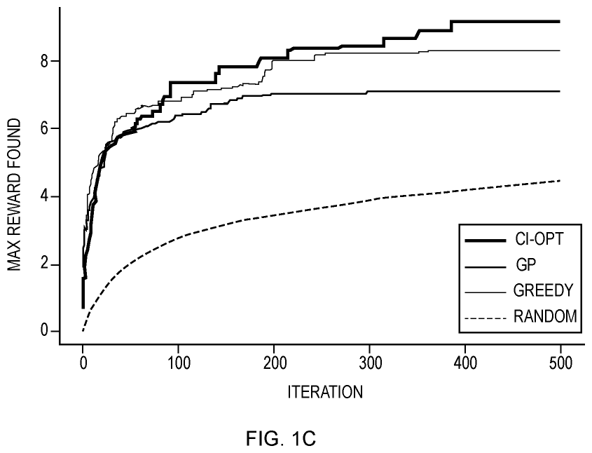

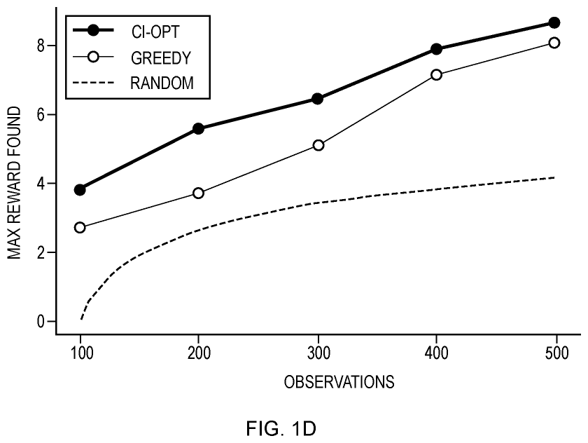

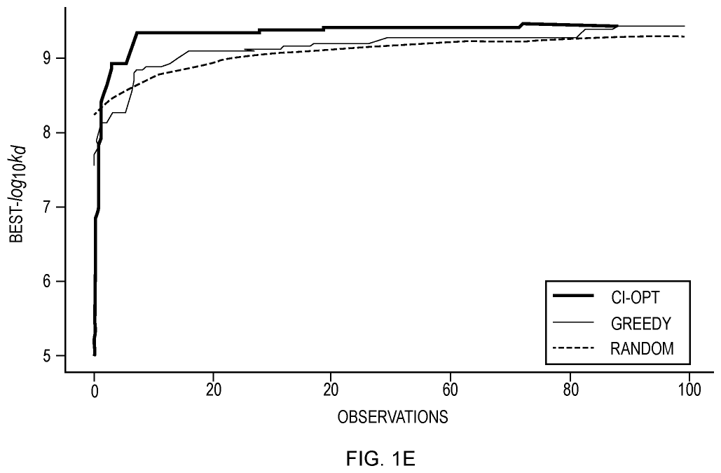

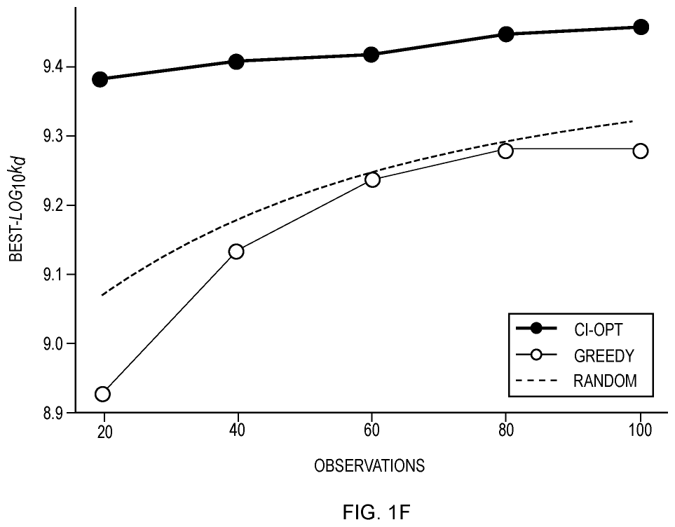

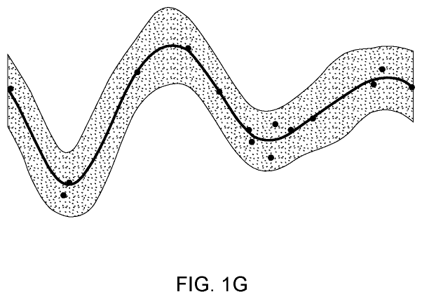

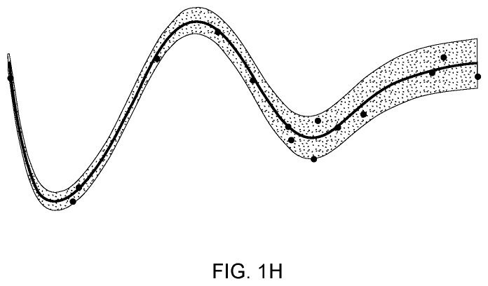

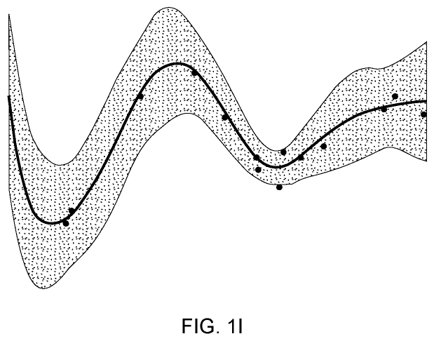

FIGS. 1G-E compare uncertainties calculated from a GP posterior, conformal inference with a neural network residual estimator, and conformal inference with scaled k-nearest neighbors (Eq. 9). The shaded region is ±2 standard deviations for the GP (FIG. 1G) and 95% for conformal inference (FIGS. 1H-I).

FIG. 1G illustrates uncertainties calculated from a GP posterior (squared exponential kernel, hyperparameters estimated by maximizing the marginal likelihood). FIGS. 1H-I illustrate uncertainties calculated from conformal inference using the training set for calibration on top of a 3-layer fully-connected neural network with sigmoid non-linearities. FIG. 1H illustrates conformal intervals generated with a neural network residual estimator for g. FIG. 1I illustrates conformal intervals generated using Eq. 9 for g with k=5 and β=0.001 for both conformal plots. Using a neural network residual estimator for g results in prediction intervals that are wider in densely sampled regions of Xₜᵣ, a problem which is exacerbated by setting Z_{c}=Zₜᵣ. Using those intervals in the acquisition function would result in the optimizer being more likely to get stuck in local optima as the weight on the uncertainty is increased.

The nearest neighbor can be determined by a distance to x in a metric space in the training set. A metric space is a set of possible sequences or data points. An example of a metric can be Levenshstein distance.

Using the conformal scores to select items to query violates the assumption of exchangeable data of GP models. Furthermore, in the small-data regime, such as at the beginning of an optimization run, the calibration scores may need to be calculated on Zₜᵣ. Therefore, the both result in prediction intervals that do not have exact, finite-sample coverage guarantees. However, these intervals remain useful for trading off exploration and exploitation during optimization.

In other words, Applicant's method includes (1) calculating conformal intervals for predictions from a fine-tuned neural network using nearest-neighbors in sequence space, and (2) Using the conformal intervals calculated in (1) to perform batch optimization.

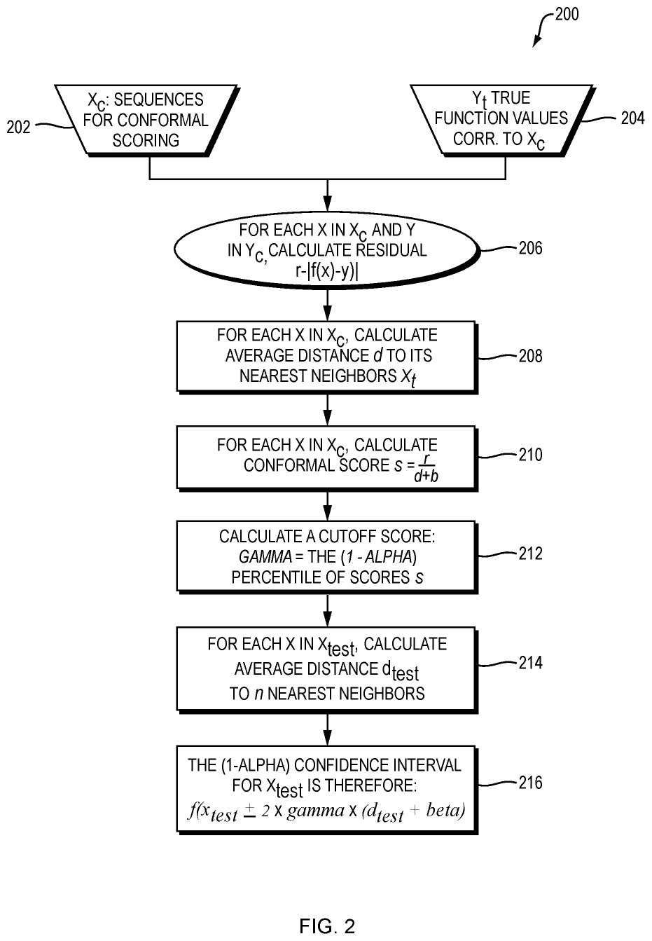

FIG. 2 is a flow diagram 200 illustrating an example embodiment calculating conformal intervals for predictions from a fine-tuned neural network using nearest-neighbors in sequence space as in the present disclosure. To calculate the conformal intervals, the method uses

a) ƒ(x): a fine-tuned neural network,

b) Xₜ: the sequences used to fine-tune ƒ(x);

c) X_{c}: the sequences used to tune the conformal scores; (202)

d) y_{c}: the true function values corresponding to X_{c}; (204)

e) n: the number of nearest neighbors to consider;

f) b: a hyperparameter;

g) alpha: the desired confidence value; and

h) Xₜₑₛₜ: new sequences to predict.

Then, for each x, y in X_{c}, y_{c}, the method calculates the residual: r=|ƒ(x)−y| (206). For each x in X_{c}, the method calculates the average distance to its n nearest neighbors in Xₜ and assign it to d (208). For x in X_{c}, calculate conformal score:

$$s = \frac{r}{d + b} . \qquad ( 210 )$$

The method then calculates a cutoff score: gamma=the (1−alpha) percentile of the scores s (212). For each x in Xₜₑₛₜ, the method calculates the average distance to the n nearest neighbors in Xₜ:d_test (214). The (1−alpha) confidence interval for Xₜₑₛₜ is therefore ƒ(xₜₑₛₜ±2×gamma×(dₜₑₛₜ+beta) (216).

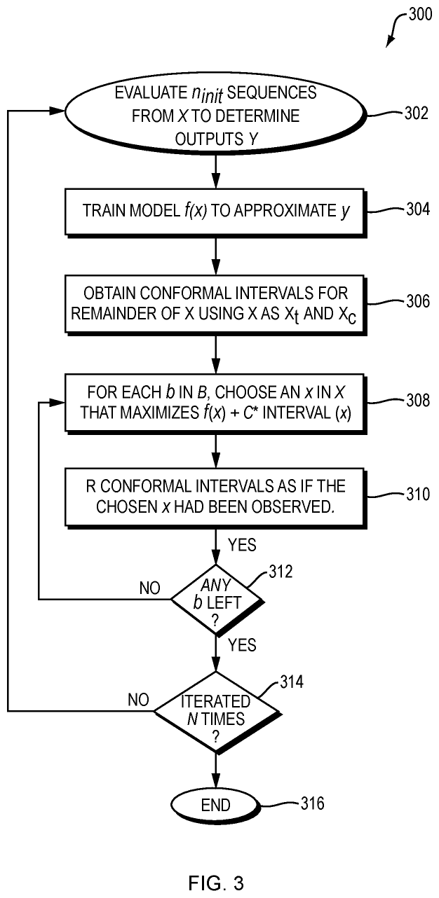

FIG. 3 is a flow diagram 300 illustrating an example embodiment of a method of batch optimization using the above conformal intervals. The method uses the following:

a) B: batch size;

b) N: number of iterations;

c) nᵢₙᵢₜ: number of initial samples;

d) X: possible sequences; and

e) C: a constant

Then, the method evaluates nᵢₙᵢₜ sequences from X to determine their outputs y (302) and train a model to ƒ(x) to approximate y (304). Using X as Xₜ and X_{c}, from the conformal inference calculations, the method obtains conformal intervals for the remainder of X (306). For each b in B, the method chooses an x in X that maximizes ƒ(x)+C*interval(x) (308) and recalculates conformal intervals as if the chosen x had been observed (310). The, the method determines whether there are any b remaining in B that 308 or 310 have not evaluated (312). If there are b remaining, the method repeats with an unevaluated b in B. Otherwise, the method determines whether more iterations are required (314), and, and after N iterations, the method ends (316).

While the above method can be used for generic data and data points, Applicant notes that it can be used to optimize the design of biopolymer sequences. Examples of biopolymer sequences include amino acid sequences, nucleotide sequences, and carbohydrate sequences.

Amino acid sequences can include canonical or non-canonical amino acids, or combinations thereof, and, furthermore, can include L-amino acids and/or D-amino acids. Amino acid sequences can also include amino acid derivatives and/or modified amino acids. Non-limiting examples of amino acid modifications include amino acid linkers, acylation, acetylation, amidation, methylation, terminal modifiers (e.g., cyclizing modifications), and N-methyl-α-amino group substitution.

Nucleotide sequences can include naturally occurring ribonucleotide or deoxyribonucleotide monomers, as well as non-naturally occurring nucleotide derivatives and analogs thereof. Accordingly, nucleotides can include, for example, nucleotides comprising naturally occurring bases (e.g., A, G, C, or T) and nucleotides comprising modified bases (e.g., 7-deazaguanosine, inosine, or methylated nucleotides, such as 5-methyl dCTP and 5-hydroxymethyl cytosine).

Examples of properties (e.g., the function values) of said biopolymer sequences (e.g., amino acid sequences) that the model analyzes are binding affinity, binding specificity, catalytic (e.g., enzymatic) activity, fluorescence, solubility, thermal stability, conformation, immunogenicity, and any other functional property of biopolymer sequences.

Described herein are devices, software, systems, and methods for evaluating input data comprising protein or polypeptide information such as amino acid sequences (or nucleic acid sequences that code for the amino acid sequences) to predict one or more specific functions or properties based on the input data. The extrapolation of specific function(s) or properties for amino acid sequences (e.g., proteins) has long been a goal of molecular biology. Accordingly, the devices, software, systems, and methods described herein leverage the capabilities of artificial intelligence or machine learning techniques for polypeptide or protein analysis to make predictions about structure and/or function. The machine learning techniques described herein enable the generation of models with increased predictive ability compared to standard non-ML approaches.

In some embodiments, input data comprises the primary amino acid sequence for a protein or polypeptide. In some cases, the models are trained using labeled data sets comprising the primary amino acid sequence. For example, the data set can include amino acid sequences of fluorescent proteins that are labeled based on the degree of fluorescence intensity. However, other types of proteins that are labeled based on other properties can be employed as well. Accordingly, a model can be trained on this data set using a machine learning method to generate a prediction of fluorescence intensity for amino acid sequence inputs. In some embodiments, the input data comprises information in addition to the primary amino acid sequence such as, for example, surface charge, hydrophobic surface area, measured or predicted solubility, or other relevant information. In some embodiments, the input data comprises multi-dimensional input data including multiple types or categories of data.

In some embodiments, the devices, software, systems, and methods described herein utilize data augmentation to enhance performance of the predictive model(s). Data augmentation entails training using similar but different examples or variations of the training data set. As an example, in image classification, the image data can be augmented by slightly altering the orientation of the image (e.g., slight rotations). In some embodiments, the data inputs (e.g., primary amino acid sequence) are augmented by random mutation and/or biologically informed mutation to the primary amino acid sequence, multiple sequence alignments, contact maps of amino acid interactions, and/or tertiary protein structure. Additional augmentation strategies include the use of known and predicted isoforms from alternatively spliced transcripts. For example, input data can be augmented by including isoforms of alternatively spliced transcripts that correspond to the same function or property. Accordingly, data on isoforms or mutations can allow the identification of those portions or features of the primary sequence that do not significantly impact the predicted function or property. This allows a model to account for information such as, for example, amino acid mutations that enhance, decrease, or do not affect a predicted protein property such as stability. For example, data inputs can comprise sequences with random substituted amino acids at positions that are known not to affect function. This allows the models that are trained on this data to learn that the predicted function is invariant with respect to those particular mutations.

The devices, software, systems, and methods described herein can be used to generate a variety of predictions. The predictions can involve protein functions and/or properties (e.g., enzymatic activity, binding properties, stability, etc.). Protein stability can be predicted according to various metrics such as, for example, thermostability, oxidative stability, or serum stability. In some embodiments, a prediction comprises one or more structural features such as, for example, secondary structure, tertiary protein structure, quaternary structure, or any combination thereof. Secondary structure can include a designation of whether an amino acid or a sequence of amino acids in a polypeptide is predicted to have an alpha helical structure, a beta sheet structure, or a disordered or loop structure. Tertiary structure can include the location or positioning of amino acids or portions of the polypeptide in three-dimensional space. Quaternary structure can include the location or positioning of multiple polypeptides forming a single protein. In some embodiments, a prediction comprises one or more functions. Polypeptide or protein functions can belong to various categories including metabolic reactions, DNA replication, providing structure, transportation, antigen recognition, intracellular or extracellular signaling, and other functional categories. In some embodiments, a prediction comprises an enzymatic function such as, for example, catalytic efficiency (e.g., specificity constant kcat/KM) or catalytic specificity.

In some embodiments, a prediction comprises an enzymatic function for a protein or polypeptide. In some embodiments, a protein function is an enzymatic function. Enzymes can perform various enzymatic reactions and can be categorized as transferases (e.g., transfers functional groups from one molecule to another), oxioreductases (e.g., catalyzes oxidation-reduction reactions), hydrolases (e.g., cleaves chemical bonds via hydrolysis), lyases (e.g., generate a double bond), ligases (e.g., joining two molecules via a covalent bond), and isomerases (e.g., catalyzes structural changes within a molecule from one isomer to another).

In some embodiments, the protein function comprises an enzymatic function, binding (e.g., DNA/RNA binding, protein binding, antibody-antigen binding, etc.), immune function (e.g., antibody, cytokine, checkpoint molecule, etc.), contraction (e.g., actin, myosin), and other functions. In some embodiments, the output comprises a value associated with the protein function such as, for example, kinetics of enzymatic function or binding. Such outputs can include metrics for affinity, specificity, and reaction rate.

In some embodiments, the machine learning method(s) described herein comprise supervised machine learning. Supervised machine learning includes classification and regression. In some embodiments, the machine learning method(s) comprise unsupervised machine learning. Unsupervised machine learning includes clustering, autoencoding, variational autoencoding, protein language model (e.g., wherein the model predicts the next amino acid in a sequence when given access to the previous amino acids), and association rules mining.

In some embodiments, a prediction comprises a classification such as a binary, multi-label, or multi-class classification. Classifications are generally used to predict a discrete class or label based on input parameters. A binary classification predicts which of two groups a polypeptide or protein belongs in based on the input. In some embodiments, a binary classification includes a positive or negative prediction for a property or function for a protein or polypeptide sequence. In some embodiments, a binary classification includes any quantitative readout subject to a threshold such as, for example, binding to a DNA sequence above some level of affinity, catalyzing a reaction above some threshold of kinetic parameter, or exhibiting thermostability above a certain melting temperature. Examples of a binary classification include positive/negative predictions that a polypeptide sequence exhibits autofluorescence, is a serine protease, or is a GPI-anchored transmembrane protein. In some embodiments, the classification is a multi-class classification. For example, a multi-class classification can categorize input polypeptides into one of more than two groups. Alternatively, a prediction can comprise a multi-label classification. Multi-class classification classifies input into one of mutually exclusive categories, whereas multi-label classification classifies input into multiple labels or groups. For example, multi-label classification may label a polypeptide as being both an intracellular protein (vs extracellular) and a protease. By comparison, multi-class classification may include classifying an amino acid as belonging to one of an alpha helix, a beta sheet, or a disordered/loop peptide sequence.

In some embodiments, a prediction comprises a regression that provides a continuous variable or value such as, for example, the intensity of auto-fluorescence or the stability of a protein. In some embodiments, the prediction comprises a continuous variable or value for any of the properties or functions described herein. As an example, the continuous variable or value can be indicative of the targeting specificity of a matrix metalloprotease for a particular substrate extracellular matrix component. Additional examples include various quantitative readouts such as target molecule binding affinity (e.g., DNA binding), reaction rate of an enzyme, or thermostability.

To show the effectiveness of the method described above, consider a comparison of CI-OPT with a nearest-neighbor conformal score to Gaussian-process based optimization on two synthetic Bayesian optimization tasks and two empirically-determined protein fitness datasets. The protein datasets have high-dimensional discrete spaces where any GP using a conventional kernel is expected to be strongly misspecified.

The following are brief descriptions of methods evaluated below:
a) GP is a Bayesian optimization with a Gaussian process surrogate function and either the UCB or MI acquisition functions.
b) GP-CI: CI-OPT with either the UCB or MI acquisition functions using a Guassian process to calculate μ_{t,CI} according to Eq. 6 and conformal inference to calculate σ_{t,CI} according to Eqs. 7 and 9.
c) NN-CI: CI-OPT with either the UCB or MI acquisition functions using a neural network to calculate μ_{t,CI} according to Eq. 6 and conformal inference to calculate σ_{t,CI} according to Eqs. 7 and 9.

The Branin, or Branin-Hoo, function is a common black-box optimization benchmark with three global optima in the 2-D square [−5,10]×[0,15]. One example black-box optimization benchmark is described in “Botorch: Programmable Bayesian Optimization in pytorch” by Balandat et al., arXiv preprint arXiv:1910.06403, 2019 (hereinafter “Botorch” or “Balandat”) having outputs normalized to have approximately mean 0 and variance 1 for numerical stability.

The Hartmann function is another common black-box optimization benchmark. Following the Botorch documentation, a 6-D version is evaluated in [0,1]⁶. The Hartmann function has six local and one global maxima.

A GB1 dataset includes measured fitness values for most sequences in a four-site site-saturation library for protein G domain B1 for a total of 160,000 sequences, as described by “Adaptation In Protein Fitness Landscapes Is Facilitated By Indirect Paths” by Wu et al. in Elife, 5:e16965, 2016 (hereinafter “Wu”). For missing sequences, values imputed by Wu can be used. The dataset is designed to capture non-linear interactions between positions and amino acids.

The FITC dataset consists of binding affinities for several thousand variants of a well-studied scFv antibody to fluorescein isothiocyanate (FITC) Adams (2016). Mutations were made in the CDR1H and CDR3H regions. A lower binding constant k_{D} indicates stronger binding, so in this case the task is to maximize −log k_{D}.

For synthetic tasks, CI-OPT is compared using the UCB acquisition function and a GP surrogate model or neural network surrogate model to GP-UCB using the same GP model. GPs for the synthetic tasks followed the defaults in Botorch (e.g., a Matérn kernel with v=2.5 with strong priors on the noise and lengthscales), and GP-UCB is performed using the reparametrized implementation in Botorch. The neural networks comprised two (2) hidden layers of dimension 256 connected with ReLU activations. Weights are optimized using Adam: A method for stochastic optimization, by Kingma et al. in arXiv preprint arXiv:1412.6980 (hereinafter “Adam” or “Kingma”) with L² weight decay set to 1e⁻³.

For each run, methods are initialized with 10 randomly-selected observations. Experiments are repeated 64 times with different initializations. Conformal inference uses β=1e⁻², Euclidean distance, and 5 nearest neighbors. GPs are retrained at each iteration. The neural nets are initially trained for 1000 minibatches and then fine-tuned with an additional 100 minibatches after each observation.

The system and method's demonstrations of effectiveness using several real-world protein datasets are further described below. For the protein tasks, CI-OPT is compared using the MI acquisition function to GP-MI under both the sequential and batch settings. GPs for the protein tasks use a square exponential kernel with hyperparameters chosen to maximize the marginal likelihood. CI-OPT uses a Transformer language model, as described in “Attention is All You Need” by Vaswani in Advances in Neural Information Processing Systems, pp. 5998-6008, 2017 (hereinafter “Vaswani”), pretrained on proteins from UniProt, as disclosed in “Biological Structure and Function Emerge from Scaling Unsupervised Learning to 250 Million Protein Sequences” by Rives in bioRxiv, pp. 622803, 2019 (hereinafter “Rives”), and then finetuned on the observations. On both datasets, CI-OPT employs the Hamming distance and five (5) nearest neighbors to calculate conformal scores. CI-OPT and greedy is repeated ten (10) times with different initial points, while GP is repeated 25 times.

As further described below, the methods are evaluated by comparing the maximum reward found by each method at iteration t instead of the average regret because in biological optimization problems, the goal is to find good rewards as quickly as possible, but there is usually not a penalty for evaluating inputs that lead to poor rewards along the way.

FIGS. 1A and 1B are graphs illustrating results for sequential optimization on the two synthetic tasks. On the 2-D Branin task, GP-UCB, GP-CI, and NN-CI all quickly find the global maximum. On the 6-D Hartmann task, GP-CI is competitive with GP-UCB, but NN-CI under-performs. However, these results were using neural networks without tuned neural network hyperparameters.

FIGS. 1C and 1E are graphs illustrating results for sequential optimization on the protein datasets. On these high dimensional and discrete spaces, NN-CI consistently outperforms GP-based methods. This performance is due to both the pretrained neural network being much more accurate than GPs and to the GP uncertainties being miscalibrated, removing their theoretical advantage.

FIGS. 1D and 1F are graphs illustrating similar results for batch optimization on the protein datasets. Optimization with large batches is extremely challenging, as each batch must balance exploration and exploitation to maximize the acquisition function. The batch size of 100 used here for GB1 is much larger than those typically seen in Bayesian optimization experiments. For example, Wilson considers batch sizes up to 16. However, 100 is a realistic batch size for protein engineering experiments.

Conformal Inference Optimization uses the prediction intervals induced by a nearest-neighbors based conformal score for regression as a drop-in replacement for GP posterior uncertainties in upper confidence bound-based acquisition functions for black-box function optimization. This method is more amenable to taking advantage of large, pre-trained neural networks in an optimization loop than traditional BO methods based on GPs. CI-OPT is competitive with GP-based Bayesian optimization on synthetic tasks and outperforms GP-based methods on two difficult protein optimization datasets.

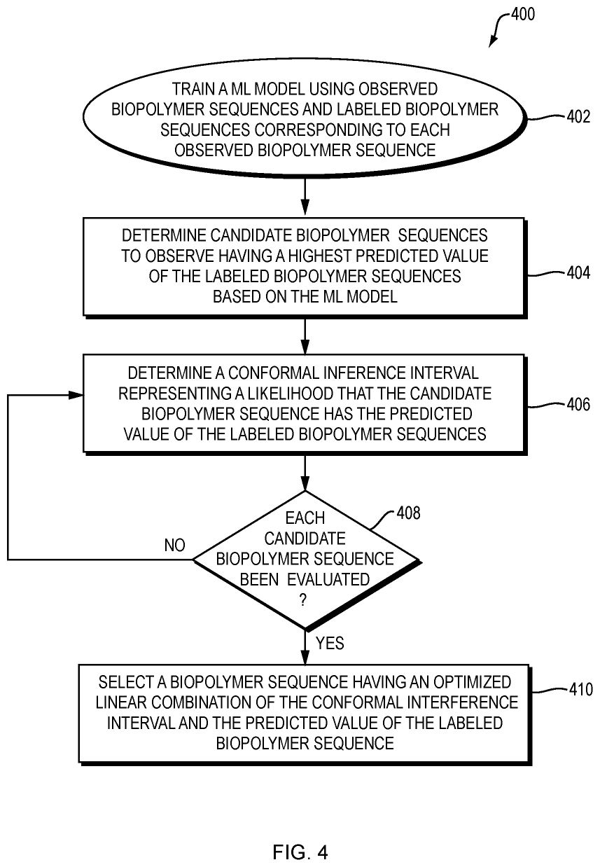

FIG. 4 is a flow diagram 400 illustrating an example embodiment of the present disclosure. In an embodiment, a computer-implemented method for optimizing design of biopolymer sequences can include training a machine learning model using an observed biopolymer sequences and labeled biopolymer sequences corresponding to each observed biopolymer sequence (402). A labeled sequence is a sequence associated with a real number measuring some property of interest. The method can further include determining a candidate biopolymer sequences to observe having a highest predicted value of the labeled biopolymer sequences based on the machine learning model (404). Candidate biopolymer sequences can include either known sequences (e.g., previously encountered, previously observed, or natural sequences) or newly designed sequences. The method can further include, for each candidate biopolymer sequence (408), determining a conformal inference interval representing a likelihood that the candidate biopolymer sequence has the predicted value of the labeled biopolymer sequences (406). The method can further include selecting at least one candidate biopolymer sequence having an optimized linear combination of the conformal inference interval and the predicted value of the labeled biopolymer sequences (410). In an embodiment, the value of a labeled sequence is number being used as its label as described above. Therefore, a predicted value of a sequence is the predicted label of the sequence. A person having ordinary skill in the machine learning art can appreciate such a definition of label. The sequence or data points are the machine learning input (x), and the prediction/measurement/optimization is the label (y).

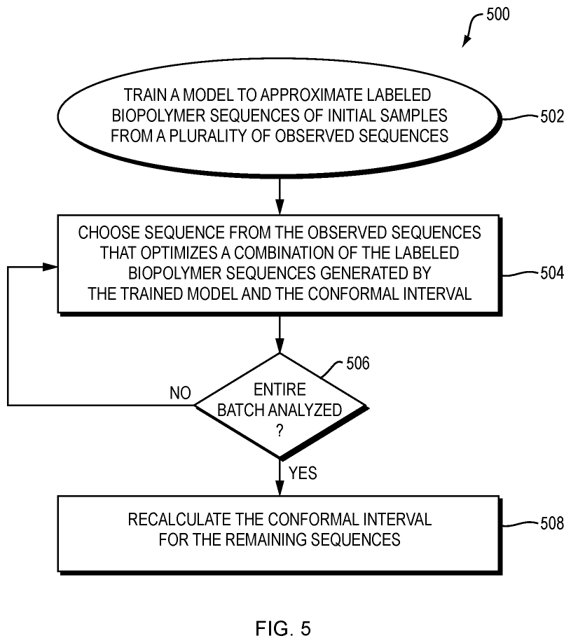

FIG. 5 is a flow diagram 500 illustrating an example embodiment of the present disclosure. In an embodiment, a computer computer-implemented method for optimizing design of biopolymer sequences and corresponding system trains a model to approximate labeled biopolymer sequences of initial samples from a plurality of observed sequences (502). The method can further include, for a particular batch of the observed sequences, choosing at least one sequence from the plurality of observed sequences that optimizes a combination of the labeled biopolymer sequences generated by the trained model and the conformal interval (504). The batches include labeled biopolymer sequences generated by a trained model and conformal interval for each observed sequence. If the entire batch has not been analyzed (506), the method chooses a next sequence (504). If the entire batch is analyzed (506), the method can further include recalculating the conformal interval for the remaining sequences (508).

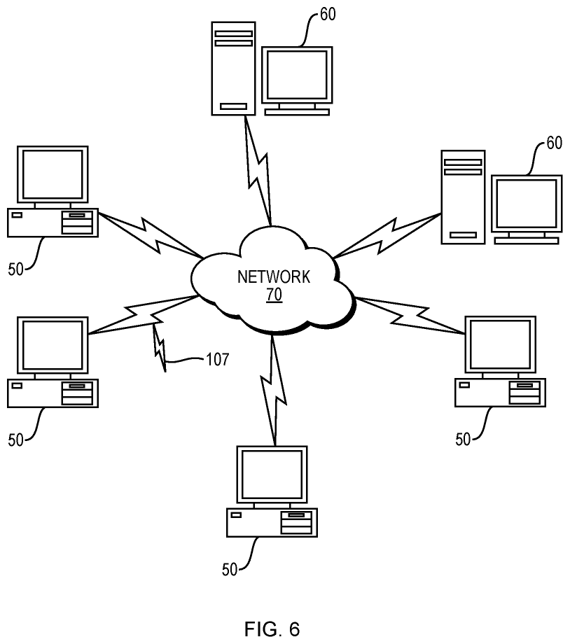

FIG. 6 illustrates a computer network or similar digital processing environment in which embodiments of the present invention may be implemented.

Client computer(s)/devices 50 and server computer(s) 60 provide processing, storage, and input/output devices executing application programs and the like. The client computer(s)/devices 50 can also be linked through communications network 70 to other computing devices, including other client devices/processes 50 and server computer(s) 60. The communications network 70 can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

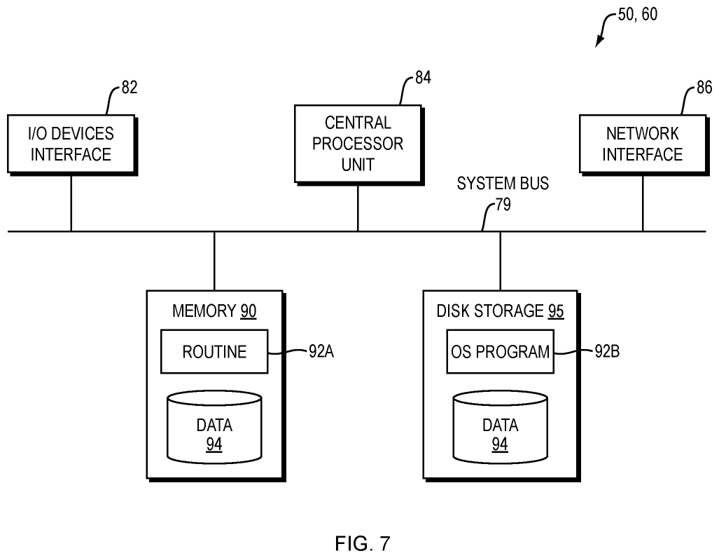

FIG. 7 is a diagram of an example internal structure of a computer (e.g., client processor/device 50 or server computers 60) in the computer system of FIG. 6. Each computer 50, 60 contains a system bus 79, where a bus is a set of hardware lines used for data transfer among the components of a computer or processing system. The system bus 79 is essentially a shared conduit that connects different elements of a computer system (e.g., processor, disk storage, memory, input/output ports, network ports, etc.) that enables the transfer of information between the elements. Attached to the system bus 79 is an I/O device interface 82 for connecting various input and output devices (e.g., keyboard, mouse, displays, printers, speakers, etc.) to the computer 50, 60. A network interface 86 allows the computer to connect to various other devices attached to a network (e.g., network 70 of FIG. 5). Memory 90 provides volatile storage for computer software instructions 92 and data 94 used to implement an embodiment of the present invention (e.g., Bayesian optimization module and conformal inference module code detailed above). Disk storage 95 provides non-volatile storage for computer software instructions 92 and data 94 used to implement an embodiment of the present invention. A central processor unit 84 is also attached to the system bus 79 and provides for the execution of computer instructions.

In one embodiment, the processor routines 92 and data 94 are a computer program product (generally referenced 92), including a non-transitory computer-readable medium (e.g., a removable storage medium such as one or more flash memory, DVD-ROM's, CD-ROM's, diskettes, tapes, etc.) that provides at least a portion of the software instructions for the invention system. The computer program product 92 can be installed by any suitable software installation procedure, as is well known in the art. In another embodiment, at least a portion of the software instructions may also be downloaded over a cable communication and/or wireless connection. In other embodiments, the invention programs are a computer program propagated signal product embodied on a propagated signal on a propagation medium (e.g., a radio wave, a microwave, an infrared wave, a laser wave, a sound wave, or an electrical wave propagated over a global network such as the Internet, or other network(s)). Such carrier medium or signals may be employed to provide at least a portion of the software instructions for the present invention routines/program 92.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A computer-implemented method for optimizing design of biopolymer sequences, the method comprising:
training a machine learning model using a plurality of observed biopolymer sequences and labeled biopolymer sequences corresponding to each observed biopolymer sequence;
determining a plurality of candidate biopolymer sequences to observe having a highest predicted value of the labeled biopolymer sequences based on the machine learning model;
for each candidate biopolymer sequence, determining a conformal inference interval representing a likelihood that the candidate biopolymer sequence has the predicted value of the labeled biopolymer sequences;
selecting at least one candidate biopolymer sequence having an optimized linear combination of the conformal inference interval and the predicted value of the labeled biopolymer sequences.

2. The computer-implemented method of claim 1, wherein the conformal inference interval includes a center value and an interval range.

3. The computer-implemented method of claim 2, wherein the center value is a mean value.

4. The computer-implemented method of claim 1, wherein the machine learning model is a neural network fine-tuned using the observed biopolymer sequences and their labels.

5. The computer-implemented method of claim 4, wherein determining the conformal inference interval is based on a second set of observed biopolymer sequences.

6. The computer-implemented method of claim 5, wherein determining the conformal inference interval further includes:
calculating a residual interval based on each output of the machine learning model for the second set of observed biopolymer sequences and corresponding labeled biopolymer sequences corresponding to each of the second set of biopolymer sequences;
for each output of the machine learning model, calculating an average distance to a plurality of nearest neighbors of the observed biopolymer sequences within a metric space; and
calculating a conformal score based on a ratio of the residual to a sum of the average distance and a constant.

7. The computer-implemented method of claim 5, wherein selecting the at least one candidate biopolymer sequence includes:
calculating an average distance in a metric space to a plurality of nearest neighbors in the metric space;
generating a confidence interval based on the at least one candidate biopolymer sequence and the average distance; and
selecting at least one candidate biopolymer sequence based on the confidence interval.

8. The method of claim 1, wherein the conformal interval is at least 50% and at most 99%.

9. The method of claim 1, wherein the biopolymer sequence includes at least one of an amino acid sequence, a nucleic acid sequence, and a carbohydrate sequence.

10. The method of claim 9, wherein the nucleic acid sequence is a deoxyribonucleic acid (DNA) sequence or ribonucleic acid (RNA) sequence.

11. The method of claim 1, wherein the predicted value is a function value of the biopolymer sequences, wherein the function is one or more of binding affinity, binding specificity, catalytic activity, enzymatic activity, fluorescence, solubility, thermal stability, conformation, immunogenicity, and any functional property of biopolymer sequences.

12. The method of claim 1, wherein selecting the at least one candidate biopolymer sequence has an increased performance compared to a Bayesian optimization without factoring the determine conformal inference interval.

13. A computer-implemented method for optimizing design of biopolymer sequences comprising:
training a model to approximate labeled biopolymer sequences of initial samples from a plurality of observed sequences;
for a particular batch of the plurality of observed sequences, having labeled biopolymer sequences generated by a trained model and conformal interval for each observed sequence, choosing at least one sequence from the plurality of observed sequences that optimizes a combination of the labeled biopolymer sequences generated by the trained model and the conformal interval; and
recalculating the conformal interval for the remaining sequences.

14. The computer-implemented method of claim 13, further comprising repeating choosing the at least one sequence and recalculating the conformal interval for each of a plurality of batches.

15. The method of claim 13, further comprising identifying an optimal number of batch experiments to run in parallel.

16. The method of claim 15, wherein identifying is based on optimizing wet-lab resources.

17. A computer-implemented method for optimizing design based on a distribution of data, the method comprising:
training a machine learning model using a plurality of observed data and labeled data corresponding to each observed data;
determining a plurality of candidate data to observe having a highest predicted value of the labeled data based on the machine learning model;
for each candidate data, determining a conformal inference interval representing a likelihood that the candidate data has the predicted value of the labeled data;
selecting at least one candidate data having an optimized linear combination of the conformal inference interval and the predicted value of the labeled data.

18. The method of any one of the preceding claims, further comprising:
providing the at least one selected biopolymer sequence to a means for synthesizing the selected biopolymer sequence.

19. The method of claim 18, wherein the at least one selected biopolymer sequence is synthesized.

20. The method of any one of the preceding claims, further comprising synthesizing the at least one selected biopolymer sequence.

21. The method of claim 18 or 20, further comprising assaying the at least one selected biopolymer sequence, e.g., in a qualitative or quantitative chemical assay.

22. A non-transitory computer readable medium storing instructions for optimizing design of biopolymer sequences thereon, wherein the instructions, when executed by a processor, cause the processor to:
train a machine learning model using a plurality of observed biopolymer sequences and labeled biopolymer sequences corresponding to each observed biopolymer sequence;
determine a plurality of candidate biopolymer sequences to observe having a highest predicted value of the labeled biopolymer sequences based on the machine learning model;
for each candidate biopolymer sequence, determine a conformal inference interval representing a likelihood that the candidate biopolymer sequence has the predicted value of the labeled biopolymer sequences;
select at least one candidate biopolymer sequence having an optimized linear combination of the conformal inference interval and the predicted value of the labeled biopolymer sequences.

23. A system for optimizing design of biopolymer sequences, the system comprising:
a processor; and
a memory with computer code instructions stored thereon, the processor and the memory, with the computer code instructions, being configured to cause the system to:
train a machine learning model using a plurality of observed biopolymer sequences and labeled biopolymer sequences corresponding to each observed biopolymer sequence;
determine a plurality of candidate biopolymer sequences to observe having a highest predicted value of the labeled biopolymer sequences based on the machine learning model;
for each candidate biopolymer sequence, determine a conformal inference interval representing a likelihood that the candidate biopolymer sequence has the predicted value of the labeled biopolymer sequences;
select at least one candidate biopolymer sequence having an optimized linear combination of the conformal inference interval and the predicted value of the labeled biopolymer sequences.

24. One or more selected biopolymer sequences, the one or more selected biopolymer sequences obtainable by the method of any one of the preceding claims.

25. The one or more selected biopolymer sequences of claim 24, wherein the one or more selected biopolymer sequences are one or more selected polypeptides sequences manufactured by the method of: culturing a host cell comprising one or more nucleic acids encoding the one or more selected polypeptide sequences, the culturing under conditions to promote synthesis of the one or more selected polypeptide sequences, and isolating the one or more selected polypeptide sequences.

26. A composition comprising the one or more selected biopolymer sequences of any one of claims 24-25, the one or more selected biopolymer sequences containing a pharmaceutically acceptable excipient.

27. A method comprising contacting the composition or selected biopolymer sequences of any one of the preceding claims with one or more of: a test compound, a biological fluid, a cell, a tissue, an organ, or an organism.
